(19) United States
(12) Patent Application Publication
(10) Pub. No.: US 2015/0376831 A1
(43) Pub. Date: Dec. 31, 2015

(54) System and Method for Cleaning and Sanitizing Mattresses

(71) Applicant: Michael Ingle, Corinth, TX (US)

(72) Inventor: Michael Ingle, Corinth, TX (US)

(21) Appl. No.: 14/290,113

(22) Filed: May 29, 2014

(51) Int. Cl. D06M 10/00; D06M 11/34; A61L 2/10; A61L 2/04; A61L 2/07; D06M 16/00; A61L 2/20

(52) U.S. Cl. CPC D06M 10/001; D06M 16/00; D06M 11/34; A61L 2/202; A61L 2/04; A61L 2/07; A61L 2/10

(57) ABSTRACT

Mattresses infested with bed bugs and/or dust mites may be cleaned and sanitized through a series of steps beginning with UV irradiation of the mattress. Next, the mattresses are subject to dry steam of a temperature of approximately 356° F. Odors are then removed from the mattress by immersing the mattress in ozone. The mattress is then vacuumed to remove debris and waste. Next the mattress is heated with infrared heaters which produce a temperature of up to 265° F. Finally, the mattress is again vacuumed to remove any remaining debris. The system is mounted on an enclosed trailer so that the system may be transported to a home, hospital, hotel or dormitory to clean and sanitize the mattresses. The system may also be contained within a building for a more permanent location.

### CROSS-REFERENCE TO RELATED APPLICATIONS

The current application is a Continuation in Part of utility patent application Ser. No. 13/507,425 filed on Jun. 27, 2012 and which is incorporated herein by reference as if fully set forth.

### STATEMENT REGARDING FEDERALLY SPONSORED RESEARCH OR DEVELOPMENT

No part of the invention disclosed herein was the subject of federally sponsored research or development.

### THE NAMES OF THE PARTIES TO A JOINT RESEARCH AGREEMENT

None

### REFERENCE TO A SEQUENCE LISTING

Not applicable.

### BACKGROUND OF THE INVENTION

1. Field of the Invention

The field of the invention disclosed herein is the cleaning and sanitizing of mattresses in both the commercial and residential setting. The invention disclosed herein is a le system incorporating ultraviolet light, dry steam, vacuum suction, ozone and infrared heat to kill and remove insects, bacteria and debris commonly found on residential and commercial bed mattresses.

2. Description of the Prior Art

Mattresses have been developed to enhance the comfort enjoyed by the individual while sleeping in bed. Mattresses are made from many different materials and may be of different sizes as well. With use, mattresses can become dirty and infested with parasites such as dust mites and bed bugs as well as bacteria such as Staphylococcus. Dust mites are microscopic insects related to other mites, and ticks, in the class Arachnida, which also includes spiders, scorpions, harvestmen (daddy-longlegs) and similar eight-legged creatures. Classification of mites is ever evolving but are currently placed in the order Astigmata, family Pyroglyphidae, and genus Dermatophagoides. The two most common species are 1) Dermatophagoides farina, the North American house dust mite and 2) Dermatophagoides pteronyssinus, the European house dust mite. Both species inhabit dwellings where humans live and where the humidity is optimal for the mite life cycle. Dust mites are infamous for causing allergies in people who sleep on mite infested mattresses. A protein in the mite fecal pellet causes asthma, or aggravates existing asthma symptoms. Dust mite material can also cause eczema whereby the skin becomes dry, itchy, red and scaly. One of the obstacles to removing mites from infested mattresses is that they are literally too small to be seen with the naked eye. Adult dust mites range in size from 250-300 microns and dust mite larvae and nymphs are even smaller making them invisible to the naked eye. Dust mites can be seen with a microscope, but such an instrument is not commonly found in the home or in a commercial setting such as a hotel, dormitory, nursing home and/or hospital. Further, microscopes require specific training on the part of the operator in order to be used properly. Dust mites can infest a used mattress in enormous numbers. Typically, the number of dust mites in an infested mattress range from 100,000 to ten million per mattress. Dust mites feed almost exclusively on dead skin cells which people shed daily by the thousands. Mattresses are the chief place where the majority of house dust mites reside. However, mites can also be found on pillows, overstuffed furniture, even rugs and carpeting. Mites can also be found where household pets sleep.

In addition to dust mites inhabiting mattresses, bed bugs can also infest mattresses. Bed bugs feed on the blood of people. The bed bug will bite the victim and ingest the victim's blood. Bed bugs can live for more than a year without feeding. The bites themselves are painless, but may leave itchy, red scars which are apparent the next morning. Typical symptoms of bed bug bites are small red spots on the skin in rows of three or more. Some people who are bitten by bed bugs demonstrate no symptoms whatsoever, while others can have severe skin reactions. People become increasingly sensitive to bed bug bites with repeated exposure to bed bug bites over time. Bed bugs are much larger in size than dust mites ranging in size from 1.5-7 millimeters. At this size, the bed bugs are visible to the naked eye and visual inspection of an infested mattress may reveal the presence of bed bugs but not dust mites because of their small size. Bed bugs live on mattresses but are only active at night. During the day the bed bug may hide, often under the buttons on mattresses, or in the seams along the edge of the mattress. Bed bugs can also be found in the slots formed by the bed frame. Bed bugs have also been found behind headboards, pictures and posters on the wall and behind peeling wallpaper, among other locations. In contrast dust mites are small enough to slip easily between the threads in the fabric of a mattress pad or the mattress itself.

In addition to bed bugs, dust mites and other insects, the mattress may be contaminated with bacteria such as those of the genus Staphylococcus. These bacteria are classified as Gram positive bacteria and are resistant to drying. Staphylococci are small in size (1-3 microns) and are present on the skin of most people in enormous numbers. The bacteria are shed from the skin of an individual onto the mattress where the bacteria can survive for some time. Bacteria such as Staphylococcus can cause a broad spectrum of illness ranging from abscesses and pustules to blood infection and death. These organisms are also becoming alarmingly resistant to many antibiotics.

Efforts have been undertaken to remove the insects and bacteria from infested mattresses. Efforts to remove bacteria have met with limited success mainly because of the enormous numbers of bacteria present on the mattress. To remove bacteria from a mattress, the mattress is usually scrubbed with an antibacterial agent which is allowed to remain in contact with the mattress until the antibacterial agent has evaporated and the mattress is dry. This method of decontamination does not eliminate the bacteria but reduces the number of bacteria by about a factor of 10.

Efforts to remove insects such as bed bugs and dust mites have centered around the use of special protective covers which are placed on the mattress to separate the insects from the sleeping individual. Clearly, this method acts only as a barrier to contact between the insects and the individual and does not kill or remove the insects from the mattress. Removal of dust mites is critical to preventing illness from dust mites because it is the digestive proteins found in the fecal pellet which causes illness and may be allergenic long after the dust mite has expired. What is needed in the art is a system and method which kills and removes insects and bacteria from a mattress.

### BRIEF DESCRIPTION OF THE INVENTION

The invention disclosed herein is a system and method which kills and removes insects and bacteria from a mattress. Specifically, the system and method which kills and removes insects and bacteria from a mattress is composed of several steps. The first step is to irradiate the mattress in UV light. The second step in the system and method disclosed herein is to clean the mattress with dry steam using distilled water of approximately 356° F. The third step is to remove any unwanted waste and debris left behind by the bed bugs and mites by a vacuum plenum. The fourth step is to heat the mattress with infrared heaters up to a temperature of 265° F. to dry any moisture created by the dry steam while killing any bed bugs and dust mites which may have survived the previous three steps. The sixth step is again a vacuum step to remove any remaining debris. The seventh step is to deodorize the mattress with an ozone immersion. The aforementioned UV lights, dry steam apparatus, vacuums, infrared heaters and ozone generator are mounted to an enclosed frame which is mounted to a box van or truck cab chassis or enclosed trailer which can be transported to a home, hospital, dormitory, hotel or other location where mattresses need to be cleaned and sanitized. Electric power is supplied to the components of the system and method by a gas or diesel powered generator. The mattress to be cleaned and sanitized is placed inside the trailer and the devices are activated and do their assigned functions automatically. In another embodiment the components of the mattress cleaning invention disclosed herein may also be housed in a building or other structure. Mattresses may be transported to the building for cleaning and sanitizing.

### BRIEF DESCRIPTION OF THE DRAWING/FIGURES

A better understanding of the system and method for cleaning and sanitizing mattresses may be had by examination of the following drawings/figures:

FIG. 1 is a side oblique view of the system and method for cleaning and sanitizing mattresses;

FIG. 2 is a side oblique view of the system and method for cleaning and sanitizing mattresses;

FIG. 3 is an elevated end view of the system and method for cleaning and sanitizing mattresses with the conveyor system removed;

FIG. 4 is a front side oblique view of the system and method for cleaning and sanitizing mattresses mounted on a vehicle;

FIG. 5 is a rear side oblique view of the system and method for cleaning and sanitizing mattresses mounted on a vehicle;

FIG. 6 is a side view of the system and method for cleaning and sanitizing mattresses mounted on a vehicle;

FIG. 7 is a top view of the system and method for cleaning and sanitizing mattresses mounted on a vehicle;

FIG. 8 is an end view of the system and method for cleaning and sanitizing mattresses mounted on a vehicle;

FIG. 9 is a partial view of the system and method for cleaning and sanitizing mattresses showing the steam manifold and heat chamber convection fans.

FIG. 10 is a view of the components of the mattress elevator system.

FIG. 11 is a view of the components of the mattress conveyor system.

### DETAILED DESCRIPTION OF THE INVENTION

The invention disclosed herein is a system and method for cleaning and sanitizing mattresses which in one embodiment consists of ultraviolet lights, dry steam, vacuum, infrared heaters, an ozone generator and an electrical power source such as a gas or diesel powered generator. In a preferred embodiment, the ultraviolet lights, dry steam, vacuum, infrared heaters and ozone generator are situated in a contained movable enclosure such as an enclosed trailer capable of being towed by a car or pickup truck. The components of the system to clean and disinfect mattresses may also be contained within a closed truck or van for transport to the work site. The electrical generator is situated outside of the truck or trailer enclosure. An opening at the end or side of the truck or trailer enclosure permits the operator to insert the mattress inside the enclosure containing the components of the cleaning and sanitizing system. The operator starts the generator which provides power to the mattress cleaning and sanitizing system and inserts the mattress. A mattress conveyor system moves the mattress to each of the components of the mattress cleaning and sanitizing system.

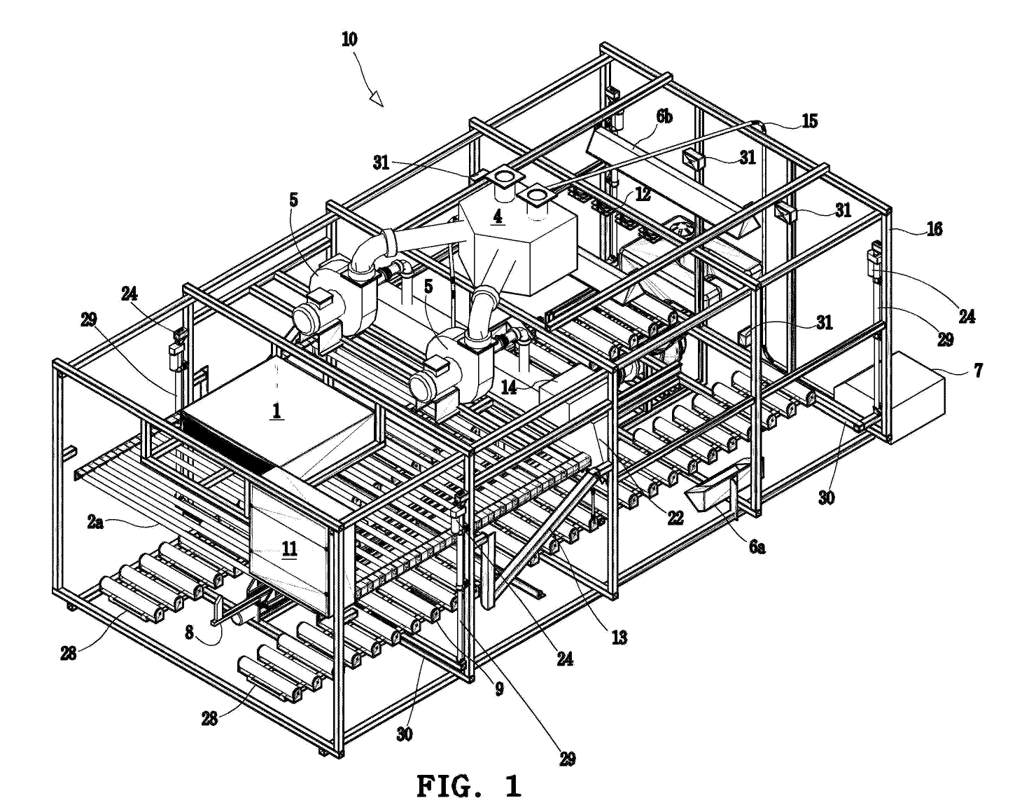

As shown in FIG. 1 the mattress cleaning and sanitizing system 10 disclosed herein is comprised of a frame 16 to which is attached or which contains the components of the cleaning and sanitizing system 10. As shown, the cleaning and sanitizing system 10 may be a stationary device which is housed in a building. In this embodiment mattresses to be cleaned and sanitized are transported to the building for treatment by the mattress cleaning and sanitizing machine 10. The mattress to be cleaned and sanitized is placed into the mattress cleaning and sanitizing machine 10 on a mattress conveyor system 8 which moves the mattress through the cleaning and sanitizing process. The components of the mattress conveyor system 8 include: a platform 30 to which is attached a plurality of rollers 28 arranged in two rows which run longitudinally through the mattress cleaning and sanitizing machine 10, a conveyor track (not shown), a screw drive (not shown), mattress holder (not shown) and mattress movement connectors (not shown). The mattress initially is exposed to the ultraviolet light from a plurality of upper ultraviolet light assemblies 2b which receive power from a UV light assembly power supply 1. The upper ultraviolet light assemblies 2b are mounted to be located approximately 2-6 inches from the mattress. In the embodiment of the system and method disclosed herein and shown in FIG. 1, 26 upper ultraviolet light assemblies 2b are mounted and configured so as to irradiate the entire mattress for approximately six minutes with ultraviolet light of approximately 254 nanometers wavelength. Bed bugs, dust mites and dust mite eggs are susceptible to UV light in approximately the 254 nanometer wavelength. The mattress is also UV irradiated by side ultraviolet lights 13. The mattress to be cleaned and sanitized is continuously moved by the mattress conveyor system 8 so that when the UV light irradiation is initiated, the mattress will start moving to the steam manifold (not shown) which will cleanse the mattress by dry steam obtained from the steam generator 7. The mattress elevator system 9 adjusts the height of the mattress conveyor system 8 so that the mattress is approximately 3 inches from the steam manifold 23. The mattress elevator system 8 is composed of a plurality of actuators 24, connected to the conveyor platform 30 by a corresponding plurality of actuator rods 29. Activation of the actuator 24 raises the conveyor platform 30 (and mattress) to the correct height. Since bed bugs are destroyed by a temperature of 113° F., the mattress to be sanitized and cleaned is dry steamed by the steam from the dry steam spray manifold (not shown) for approximately 6 minutes. The steam is created by a steam generator 7 and is transported to the dry steam spray manifold (not shown) by a steam supply line 15. Offensive odors are removed from the mattress by ozone obtained from an ozone generator 14. The ozone generator 14 produces ozone and ozone is dispersed onto the mattress to saturate the interior of the mattress cleaning and sanitizing system 10. As shown in this embodiment, two separate vacuum fans 5 each developing 1.5 horsepower are used to provide vacuum. These vacuum fans 5 create a volumetric air flow of 1280 cubic feet per minute each. The mattress is vacuumed by the vacuum plenums 22 for removal of waste and debris from the surface of the mattress which is then trapped and contained in a filter assembly located in a vacuum exhaust plenum 4. Upon leaving the vacuum plenum 22, the mattress is moved by the mattress conveyor system 8 to a position underneath the upper infrared heaters 6b and above the lower infrared heaters 6a. The infrared heaters 6a, 6b generate a heat of up to 265° F. to evaporate any moisture remaining from the dry steam cycle and kill any remaining insects. The mattress is exposed to the heat of the infrared heaters 6a, 6b for approximately 6 minutes. The mattress elevator system 9 position the mattress conveyor system 8 so that the mattress is approximately 18 inches from the upper infrared heaters 6b lower infrared heaters 6a. The heat from the upper infrared heaters 6b and lower infrared heaters 6a is dispersed over the mattress by a plurality of heat chamber convection fans 12. A plurality of thermal limiters 31 positioned on the sides and end of the frame 16 open the circuit energizing the heater relay coils until the wall temperature falls below 150° F. at which time the limits resent and reenergize the infrared heater circuit. At the conclusion of the infrared heating cycle, the conveyor system 8 moves the mattress from the infrared heaters 6a, 6b to the vacuum plenum 22 for removal of any remaining debris. After passing under the vacuum plenums 22, the mattress is moved to the starting position underneath the upper UV light assemblies 2b and above the lower UV light assemblies 2a. The mattress elevator system 9 lowers the mattress for removal from the mattress cleaning and sanitizing system 10. The power is turned off and the mattress is removed from the mattress cleaning and sanitizing system 10 enclosure. The entire cleaning and disinfecting cycle takes about 15 minutes. At the conclusion of the cleaning and sanitizing procedure, the mattress is cleaner and free of observable bed bugs or dust mites.

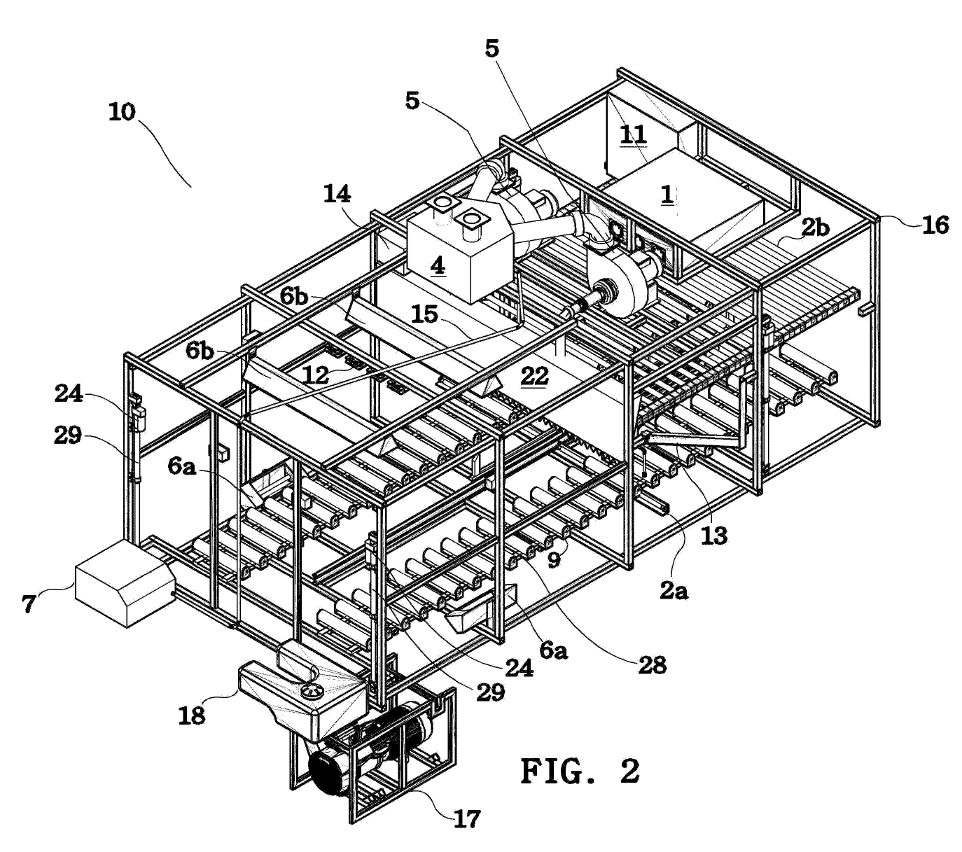

Another perspective the of the mattress cleaning and sanitizing system 10 is shown by viewing the end opposite from the end of the mattress cleaning and sanitizing system 10 in which the mattress is inserted into the mattress cleaning and sanitizing system 10 as shown in FIG. 2. Attached to the frame 16 is an electrical generator 17 which receives fuel from the electrical generator fuel tank 18. The electrical generator 17 provides electricity to operate the various components of the mattress cleaning and sanitizing system 10. Optionally, electricity could be provided by the electrical power supply of a building in which the mattress cleaning and sanitizing system is contained. A steam generator 7 attached to the frame 16 provides steam for the steam manifold (not shown) through a steam supply line 15. The mattress receives UV irradiation by a plurality of lower UV light assemblies 2a, upper UV lights assemblies 2b and side UV light assemblies 13 which receive power from a UV light assembly power supply 1. The mattress to be treated is cleaned by dry steam from a steam manifold (not shown) which receives steam from a steam generator 7 through a steam supply line 15. Odors are removed from the mattress by dispersing ozone around the mattress from an ozone generator 14. Debris from the mattress is removed by a vacuum through a vacuum plenum 22 from a plurality of vacuum fans 5. The debris removed by the vacuum is collected and isolated from the mattress cleaning and sanitizing system 10 through a vacuum exhaust plenum/filter assembly 4. A plurality of upper infrared heaters 6b and lower infrared heaters 6a provides heat which is distributed over the mattress by heat chamber convection fans 12. The infrared heaters 6a, 6b collectively generate up to 19,114 BTU's per hour of heat which produces temperatures of up to 265 degrees. The infrared heaters 6a, 6b will evaporate any moisture created by the dry steam while also killing any surviving bed bugs and dust mites. The mattress is moved through the mattress cleaning and sanitizing system 10 by a mattress conveyor system 8 on the rollers 28 and raised or lowered to the proper height by a mattress elevator system 9 by actuators 24 connected by actuator rods 29 to the conveyor system 8.

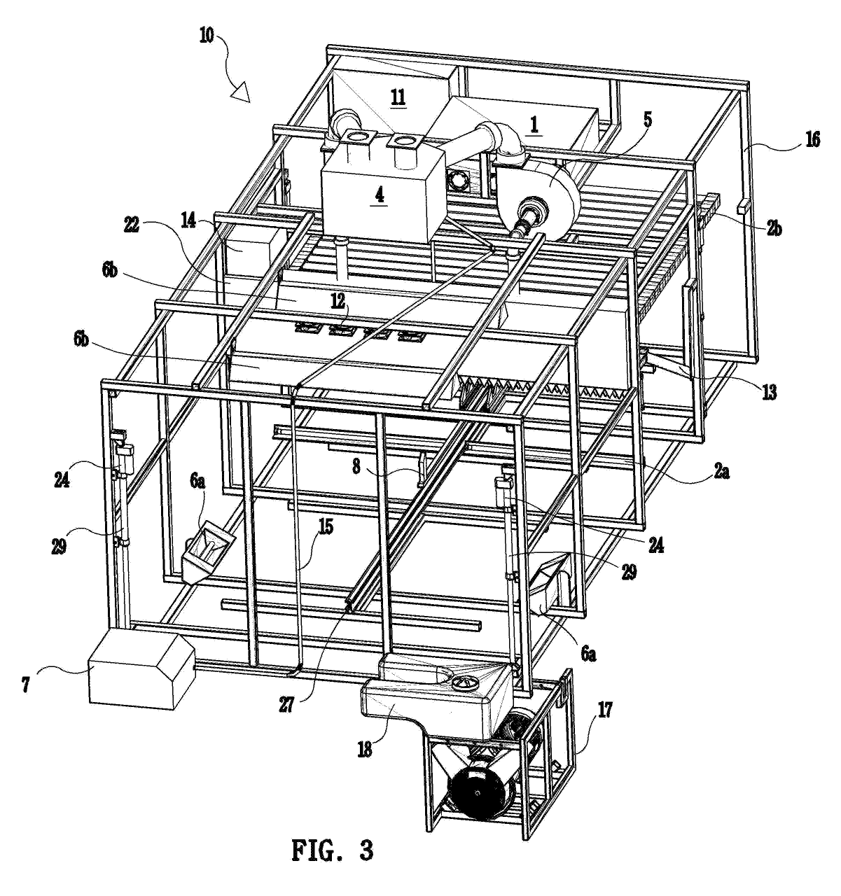

Another view of the mattress cleaning and sanitizing system 10 with some of the mattress conveyor system 8 removed for clarity is shown in FIG. 3. Attached to the frame 16 of the mattress cleaning and sanitizing system 10 are: 1) a UV light assembly power supply 1, 2) lower UV light assemblies 2a, 3) upper UV light assemblies 2b, 4) side UV light assemblies 13, 5) a vacuum exhaust plenum 4, 6) vacuum fans 5, 7) vacuum plenum 22, 8) ozone generator 14, 9) lower infrared heaters 6a, 10) upper infrared heaters 6b, 11) steam generator 7, 12) steam supply line 15, 13) electrical generator 17, 14) electrical generator fuel supply 18, 15) heat chamber convection fans 12, 16) conveyor track 27 of the mattress conveyor system 8 and 17) the actuators 24 connected to the conveyor platform by corresponding actuator rods 29 of the mattress elevator system 9.

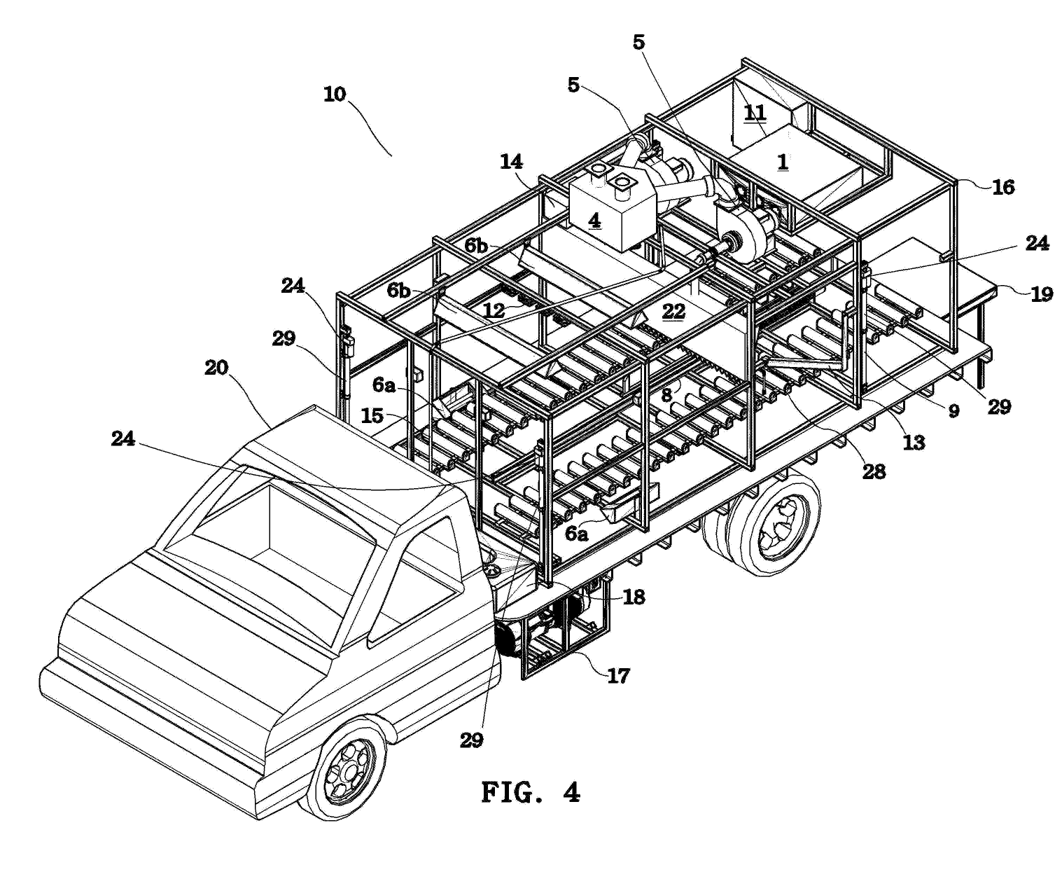

In another embodiment, the mattress cleaning and sanitizing system 10 may be attached to a vehicle such as a truck 20 to take the mattress cleaning and sanitizing system 10 to the facility which needs mattress cleaning as shown in FIG. 4. The various components of the mattress cleaning and sanitizing system 10 are attached to the frame 16 which is affixed to a truck 20. The components of the mattress cleaning and sanitizing system 10 shown include: 1) UV light assembly power supply 1, 2) power distribution panel 11, 3) vacuum fans 5, 4) vacuum exhaust plenum 4, 5) vacuum plenum 22, 6) side UV light assembly 13, 7) the plurality of rollers 28 of the mattress conveyor system 8, 8) actuators 24 and actuator rods 29 of the mattress elevator system 9, 9) lower infrared heaters 6a, 10) upper infrared heaters 6b, 11) heat chamber convection fans 12, 12) steam supply line 15, 13) electrical generator 17, electrical generator fuel tank 18, 14), a pre-treatment table 19. The pre-treatment table 19 provides a work surface on which the mattress can be placed to enable the operator to focus on specific problems on the mattress.

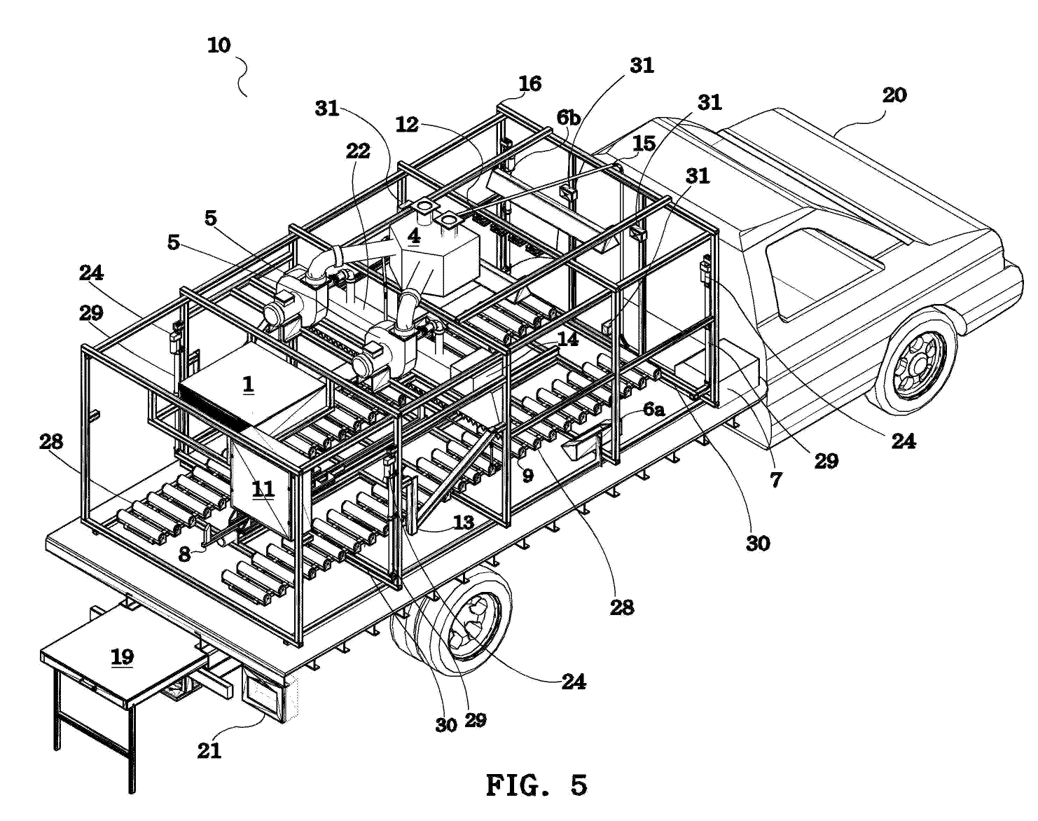

Another view of the mattress cleaning and sanitizing system 10 affixed to a truck 20 is shown in FIG. 5. The components of the mattress cleaning and sanitizing system 10 attached through the frame 16 to a truck 20 and shown are the: 1) mattress conveyor system 8 including the conveyer platform 30 and rollers 28, 2) mattress elevator system 9, 3) mattress elevator system 9 including the actuators 24 and actuator rods 29 connected to the mattress conveyor platform 30, 4) power distribution panel 11, 4) ultraviolet light power supply 1, 5) side UV light assembly 13, 6) lower infrared heater 6a, 7) upper infrared heater 6b, 8) vacuum fans 5, 9) vacuum exhaust plenum 4, 10) vacuum plenum 22, 11) ozone generator 14, 12) steam generator 7, 13) steam supply line, 15 14) pre-treatment table 19 and 15) thermal limiters 31. The components of the mattress cleaning and sanitizing system 10 may be operated through the master control panel 21 located at the rear of the truck and in a location convenient to the pre-treatment table 19.

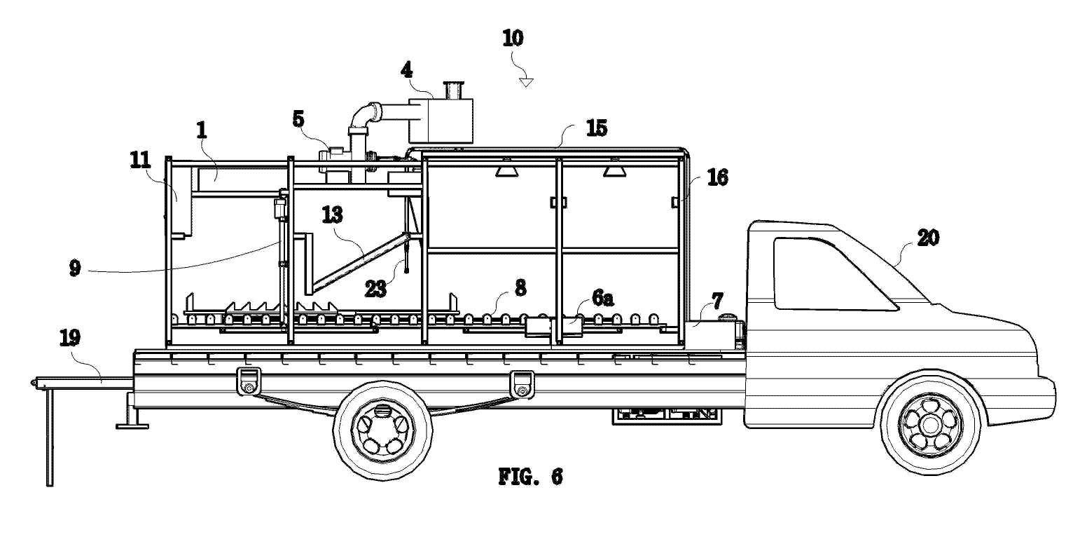

A side view of the mattress cleaning and sanitizing system 10 attached to a truck 20 is shown in FIG. 6. The components of the mattress cleaning and sanitizing system 10 shown include the pre-treatment table 19, master control panel 21, power distribution panel 11, ultraviolet power supply 1, side UV light assembly 13, vacuum fan 5, vacuum exhaust plenum/filter assembly 4, steam manifold 23, steam supply line 15, steam generator 7, ozone generator 14, mattress conveyor system 8, mattress elevator system 9, lower infrared heater 6a and upper infrared heaters 6b.

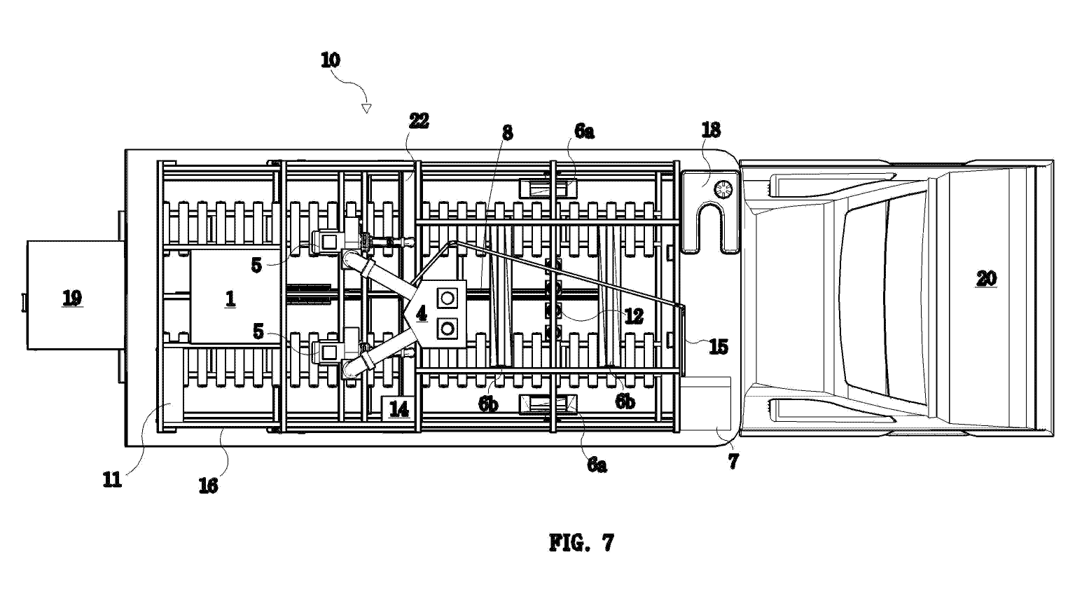

FIG. 7 is a top view of the mattress cleaning and sanitizing system 10 attached to the frame 16 and affixed to a truck 20 showing the steam generator 7, steam supply line 15, electric generator fuel tank 18, lower infrared heaters 6a, upper infrared heaters 6b, heat chamber convection fans 12, mattress conveyor system 8, mattress elevator system 9, vacuum exhaust plenum/filter assembly 4, vacuum fans 5, vacuum plenum 22, ozone generator 14, UV light power supply 1, power distribution panel 11 and pre-treatment table 19.

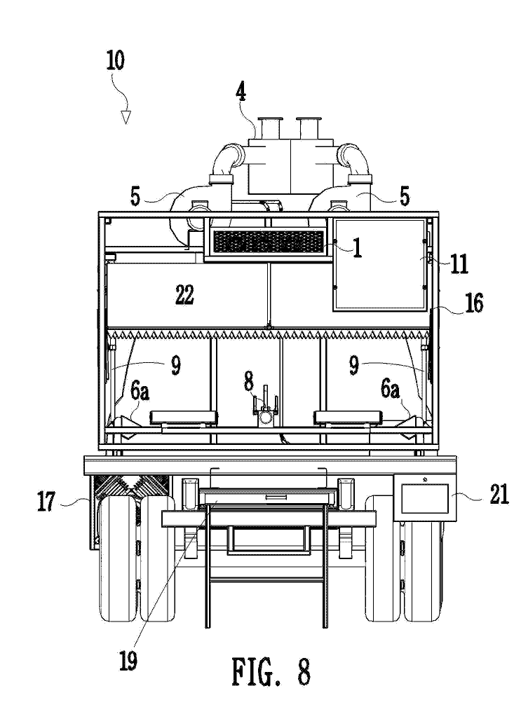

FIG. 8 is a rear view of the mattress cleaning and sanitizing system 10 attached to a frame 16 and affixed to a truck 20 showing the pre-treatment table 19, master control panel 21, electrical generator 17, lower infrared heaters 6a, mattress conveyor system 8, mattress elevator system 9, vacuum plenum 22, power distribution panel 11, UV light assembly power supply 1, vacuum fans 5 and vacuum plenum exhaust 4.

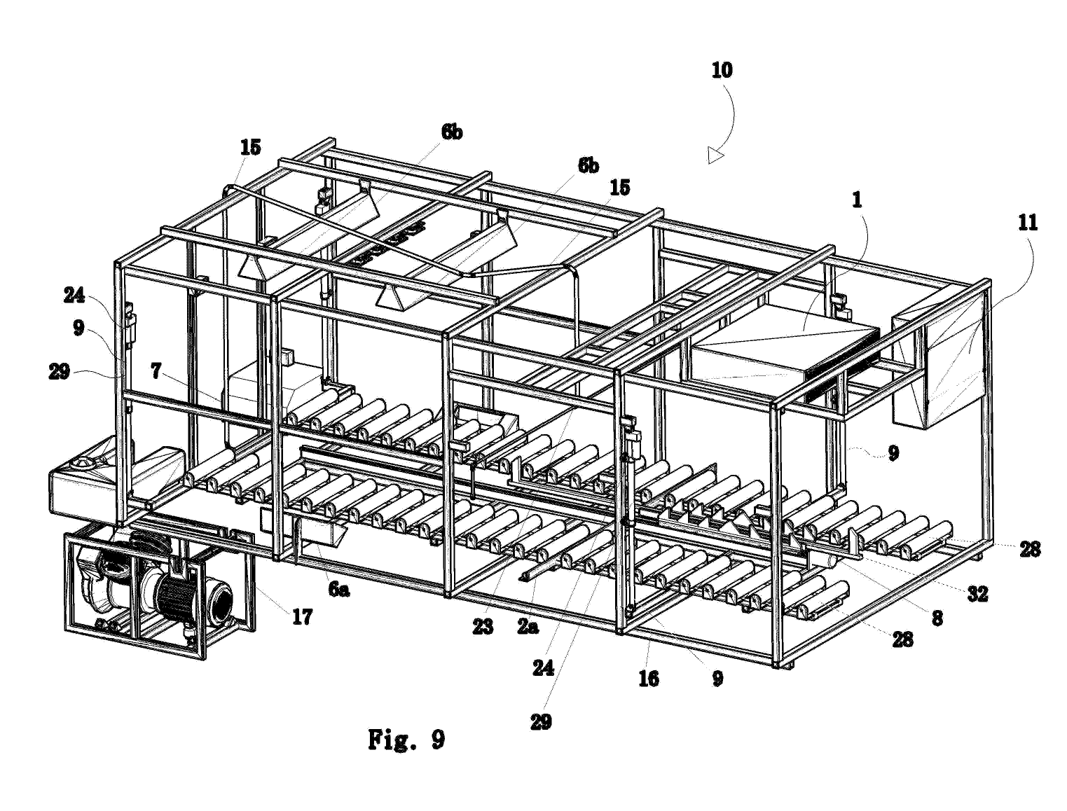

A better view of the steam manifold 23 is shown in FIG. 9 in which many of the components of the mattress cleaning and sanitizing system 10 have been removed. Also shown are the frame 16 to which are attached the UV assembly power supply 1, mattress conveyor system 8 including the rollers 28, conveyor track 27 and mattress holder 32, mattress elevator system 9 including the actuators 24 and actuator rods 29, lower infrared heaters 6a, upper infrared heaters 6b, steam supply line 15, steam generator 7 and electrical generator 17.

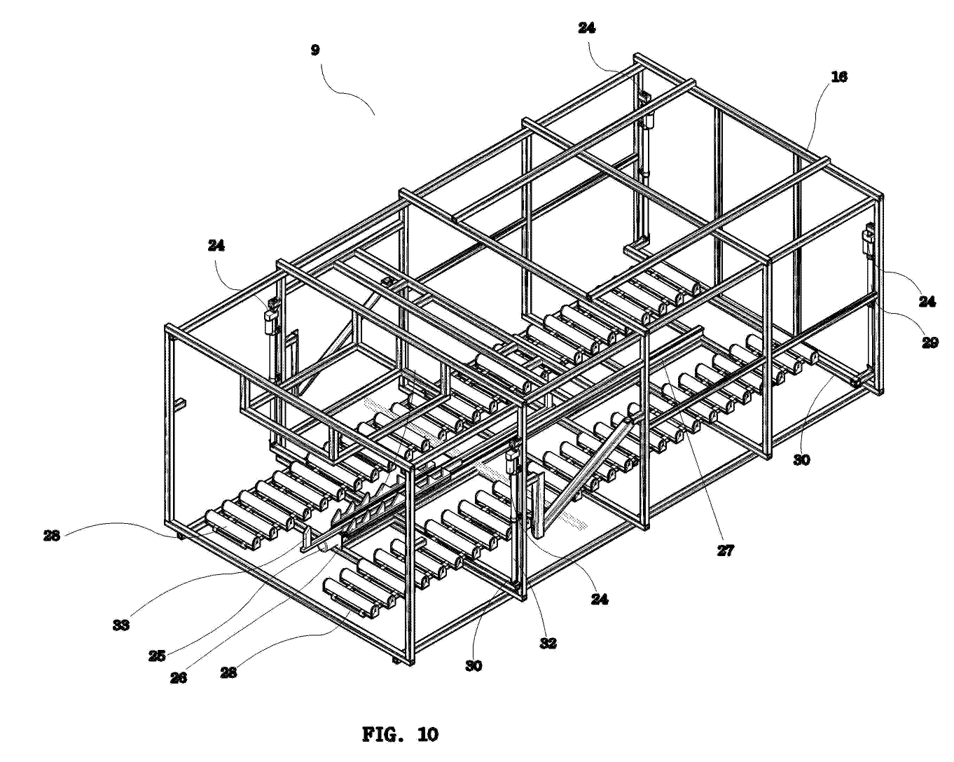

The mattress conveyor system 8 and mattress elevator system 9 are shown in FIG. 10. Both mattress conveyor system 8 and mattress elevator system 9 are connected to the frame 16. A mattress to be cleaned and sanitized is placed on the mattress conveyor system and held in position by a plurality of mattress holders 32. The mattress is moved along the conveyor track 27 by a screw drive 25 which moves a plurality of mattress holders 26 in contact with the mattress. The plurality of mattress holders 26 driven by the screw drive 25 travels along the conveyor track 27 and moves the mattress from one station to another for cleaning and sanitizing. In another embodiment the mattress holders 26 may be moved along the conveyor track by a number of methods including, by way of example, a cable/pulley system.

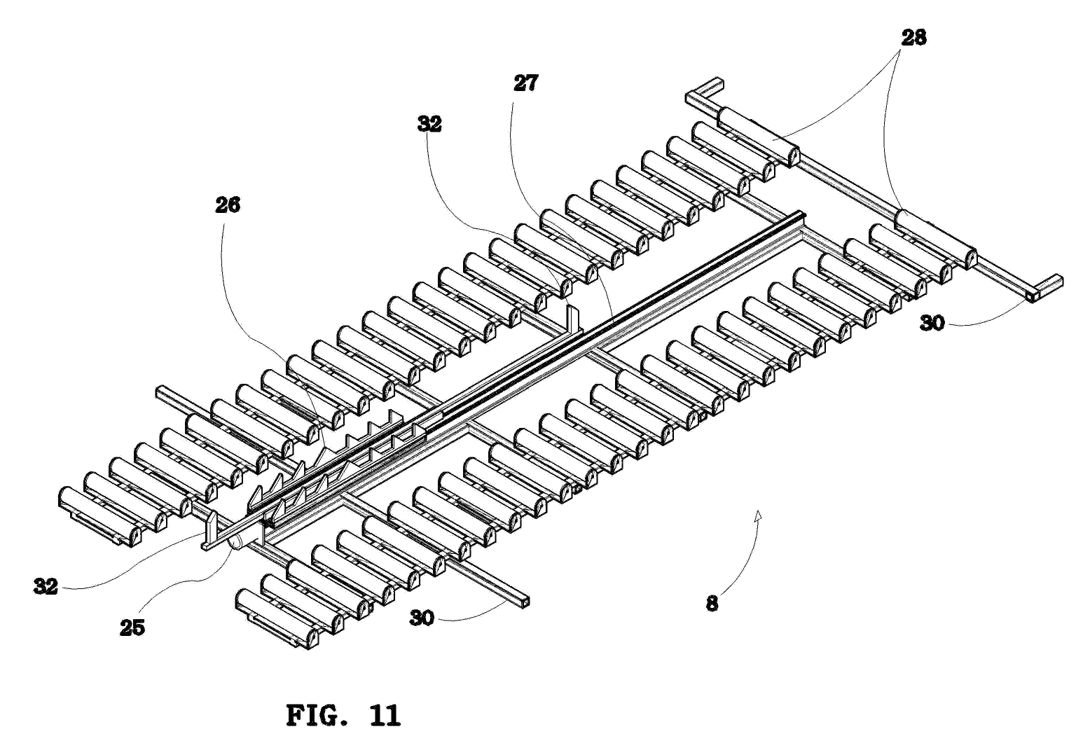

A better view of the mattress conveyor system 8 may be seen in FIG. 11 in which most of the components of the mattress cleaning and sanitizing system have been removed. Two parallel rows of rollers 28 upon which the mattress travels are attached to the conveyor platform 30. A conveyor track 27 is positioned between the two rows of rollers 28. A pair of parallel mattress movement connectors 26 move along the conveyor track 27 driven by a screw drive 25. The mattress to be cleaned and sanitized is held in place in connection with the mattress movement connectors 26 by a pair of mattress holders 32 located on a travel bar 33 running between the mattress movement connectors 26 and connected to the screw drive 25. The length of the travel bar 33 is adjustable so that the mattress holders 32 may be fitted to different size mattresses.

The system and method for cleaning and sanitizing mattresses disclosed herein is susceptible to many different embodiments. The size and number of the various components can vary to accomplish the same goals with respect to other items constructed from or covered by a fabric such as pillows, blankets, chair and sofa cushions, pet beds, rugs, draperies, clothing, luggage and a myriad of other items. The fabric from which the items to be cleaned and sanitized are constructed or covered may be of natural components such as wool, cashmere, mohair, rabbit and other animal fur, cotton, flax, hemp and other plant materials as well as artificial components such as nylon, rayon, polyester, plastic, vinyl, glass, metal, acrylic and spandex. All of these other embodiments are incorporated into this specification as limited by the claims appended hereto.

I claim:

1. A system for sanitizing and cleaning mattresses infested with insects and bacteria comprising:
a source of electrical power;
a plurality of UV light assemblies which emit UV light in approximately the wavelength of approximately 254 nanometers;
a source of dry steam which steams said mattress with steam in the range of approximately 300-380° F.;
a plurality of vacuum sources which are nearly touching said mattress and which produce a volumetric air flow of approximately 1280 cubic feet per minute each;
an ozone source;
a source of infrared heat which generates heat of approximately 265° F.;
a plurality of heat chamber convection fans;
a conveyor system to move a mattress from said UV light assemblies to said source of dry steam to said source of ozone to said source of vacuum to said source of infrared heat to said source of vacuum;
a mattress elevator system to position said mattress at the correct height under said UV light assemblies, said source of dry steam, said source of ozone, said source of vacuum and said source of infrared heat.

2. The system for sanitizing and cleaning mattresses infested with insects and bacteria as defined in claim 1 wherein said insect is a bed bug.

3. The system for sanitizing and cleaning mattresses infested with insects and bacteria as defined in claim 1 wherein said insect is a dust mite.

4. A method for sanitizing and cleaning mattresses infested with insects and bacteria comprising steps for:
irradiating said mattress with UV light located approximately 2-6 inches from said mattress of a frequency of approximately 254 nanometers for approximately 6 minutes from a plurality of UV light sources;
steaming said mattress with steam located approximately 3 inches from said mattress at a temperature of approximately 356° F. for approximately 6 minutes;
dispersing ozone around said mattress;
vacuuming said mattress with a plurality of vacuums which are nearly touching said mattress and which produce a volumetric air flow of approximately 1280 cubic feet per minute each;
heating for approximately 6 minutes said mattress with infrared heat of approximately 265° F. located approximately 18 inches away from said mattress for approximately 6 minutes;
vacuuming said mattress with a plurality of vacuums which are nearly touching said mattress and which produce a volumetric air flow of approximately 1280 cubic feet per minute each.

5. The method for cleaning and sanitizing mattresses infested with insects and bacteria as defined in claim 4 wherein said insect is a bed bug.

6. The method for cleaning and sanitizing mattresses infested with insects and bacteria as defined in claim 4 wherein said insect is a dust mite.

7. A system for sanitizing and cleaning mattresses comprising:
a source of electrical power;
a plurality of UV light assemblies which emit UV light in approximately the wavelength of approximately 254 nanometers;
a source of dry steam which steams said mattress with steam in the range of approximately 300-380° F.;
a plurality of vacuum sources which are nearly touching said mattress and which produce a volumetric air flow of approximately 1280 cubic feet per minute each;
an ozone source;
a source of infrared heat which generates heat of approximately 265° F.;
a plurality of heat chamber convection fans;
a conveyor system to move a mattress from said UV light assemblies to said source of dry steam to said source of ozone to said source of vacuum to said source of infrared heat to said source of vacuum;
a mattress elevator system to position said mattress at the correct height under said UV light assemblies, said source of dry steam, said source of ozone, said source of vacuum and said source of infrared heat.

8. A method for sanitizing and cleaning mattresses comprising steps for:
irradiating said mattress with UV light located approximately 2-6 inches from said mattress of a frequency of approximately 254 nanometers for approximately 6 minutes from a plurality of UV light sources;
steaming said mattress with steam located approximately 3 inches from said mattress at a temperature of approximately 356° F. for approximately 6 minutes;
dispersing ozone around said mattress;
vacuuming said mattress with a plurality of vacuums which are nearly touching said mattress and which produce a volumetric air flow of approximately 1280 cubic feet per minute each;
heating for approximately 6 minutes said mattress with infrared heat of approximately 265° F. located approximately 18 inches away from said mattress for approximately 6 minutes;
vacuuming said mattress with a plurality of vacuums which are nearly touching said mattress and which produce a volumetric air flow of approximately 1280 cubic feet per minute each.

9. A system for sanitizing and cleaning items constructed from a fabric infested with insects and bacteria comprising:
a source of electrical power;
a plurality of UV light assemblies which emit UV light in approximately the wavelength of approximately 254 nanometers;
a source of dry steam which steams said item constructed from a fabric with steam in the range of approximately 300-380° F.;
a plurality of vacuum sources which are nearly touching said item constructed from a fabric and which produce a volumetric air flow of approximately 1280 cubic feet per minute each;
an ozone source;
a source of infrared heat which generates heat of approximately 265° F.;
a plurality of heat chamber convection fans;
a conveyor system to move said item constructed from a fabric from said UV light assemblies to said source of dry steam to said source of ozone to said source of vacuum to said source of infrared heat to said source of vacuum;
an elevator system to position said item constructed from a fabric at the correct height under said UV light assemblies, said source of dry steam, said source of ozone, said source of vacuum and said source of infrared heat.

10. The system for sanitizing and cleaning items constructed from a fabric infested with insects and bacteria as defined in claim 9 wherein said insect is a bed bug.

11. The system for sanitizing and cleaning items constructed from a fabric infested with insects and bacteria as defined in claim 9 wherein said insect is a dust mite.

12. The system for sanitizing and cleaning items constructed from a fabric infested with insects and bacteria as defined in claim 9 wherein said item constructed from a fabric is a pillow.

13. The system for sanitizing and cleaning items constructed from a fabric infested with insects and bacteria as defined in claim 9 wherein said item constructed from a fabric is a seat cushion.

14. The system for sanitizing and cleaning items constructed from a fabric infested with insects and bacteria as defined in claim 9 wherein said item constructed from a fabric is a blanket.

15. The system for sanitizing and cleaning items constructed from a fabric infested with insects and bacteria as defined in claim 9 wherein said item constructed from a fabric is a pet bed.

15. The system for sanitizing and cleaning items constructed from a fabric infested with insects and bacteria as defined in claim 9 wherein said item constructed from a fabric is a rug.

16. The system for sanitizing and cleaning items constructed from a fabric infested with insects and bacteria as defined in claim 9 wherein said item constructed from a fabric is an article of clothing.

17. The system for sanitizing and cleaning items constructed from a fabric infested with insects and bacteria as defined in claim 9 wherein said item constructed from a fabric is an article of luggage.

18. A system for sanitizing and cleaning items constructed from a fabric comprising:
a source of electrical power;
a plurality of UV light assemblies which emit UV light in approximately the wavelength of approximately 254 nanometers;
a source of dry steam which steams said item constructed from a fabric with steam in the range of approximately 300-380° F.;
a plurality of vacuum sources which are nearly touching said item constructed from a fabric and which produce a volumetric air flow of approximately 1280 cubic feet per minute each;
an ozone source;
a source of infrared heat which generates heat of approximately 265° F.;
a plurality of heat chamber convection fans;
a conveyor system to move said item constructed from a fabric from said UV light assemblies to said source of dry steam to said source of ozone to said source of vacuum to said source of infrared heat to said source of vacuum;
an elevator system to position said item constructed from a fabric at the correct height under said UV light assemblies, said source of dry steam, said source of ozone, said source of vacuum and said source of infrared heat.

19. The system for sanitizing and cleaning items constructed from a fabric as defined in claim 18 wherein said item constructed from a fabric is a pillow.

20. The system for sanitizing and cleaning items constructed from a fabric as defined in claim 18 wherein said item constructed from a fabric is a seat cushion.

21. The system for sanitizing and cleaning items constructed from a fabric as defined in claim 18 wherein said item constructed from a fabric is a blanket.

22. The system for sanitizing and cleaning items constructed from a fabric as defined in claim 18 wherein said item constructed from a fabric is a pet bed.

23. The system for sanitizing and cleaning items constructed from a fabric as defined in claim 18 wherein said item constructed from a fabric is a rug.

24. The system for sanitizing and cleaning items constructed from a fabric as defined in claim 18 wherein said item constructed from a fabric is an article of clothing.

25. The system for sanitizing and cleaning items constructed from a fabric as defined in claim 18 wherein said item constructed from a fabric is an article of luggage.

26. A method for sanitizing and cleaning items constructed from a fabric infested with insects and bacteria comprising steps for:
irradiating said item constructed from a fabric with UV light located approximately 2-6 inches from said item constructed from a fabric of a frequency of approximately 254 nanometers for approximately 6 minutes from a plurality of UV light sources;
steaming said item constructed from a fabric with steam located approximately 3 inches from said item constructed from a fabric at a temperature of approximately 356° F. for approximately 6 minutes;
dispersing ozone around said item constructed from a fabric;
vacuuming said item constructed from a fabric with a plurality of vacuums which are nearly touching said item constructed from a fabric and which produce a volumetric air flow of approximately 1280 cubic feet per minute each;
heating for approximately 6 minutes said item constructed from a fabric with infrared heat of approximately 265° F. located approximately 18 inches away from said item constructed from a fabric for approximately 6 minutes;

27. The method for cleaning and sanitizing items constructed from a fabric infested with insects and bacteria as defined in claim 26 wherein said insect is a bed bug.

28. The method for cleaning and sanitizing items constructed from a fabric infested with insects and bacteria as defined in claim 26 wherein said insect is a dust mite.

29. The method for sanitizing and cleaning items constructed from a fabric infested with insects and bacteria as defined in claim 26 wherein said item constructed from a fabric is a pillow.

30. The method for sanitizing and cleaning items constructed from a fabric infested with insects and bacteria as defined in claim 26 wherein said item constructed from a fabric is a seat cushion.

31. The method for sanitizing and cleaning items constructed from a fabric infested with insects and bacteria as defined in claim 26 wherein said item constructed from a fabric is a blanket.

32. The method for sanitizing and cleaning items constructed from a fabric infested with insects and bacteria as defined in claim 26 wherein said item constructed from a fabric is a pet bed.

33. The method for sanitizing and cleaning items constructed from a fabric infested with insects and bacteria as defined in claim 26 wherein said item constructed from a fabric is a rug.

34. The method for sanitizing and cleaning items constructed from a fabric infested with insects and bacteria as defined in claim 26 wherein said item constructed from a fabric is an article of clothing.

35. The method for sanitizing and cleaning items constructed from a fabric infested with insects and bacteria as defined in claim 26 wherein said item constructed from a fabric is an article of luggage.

36. A method for sanitizing and cleaning items constructed from a fabric comprising steps for:
irradiating said item constructed from a fabric with UV light located approximately 2-6 inches from said item constructed from a fabric of a frequency of approximately 254 nanometers for approximately 6 minutes from a plurality of UV light sources;
steaming said item constructed from a fabric with steam located approximately 3 inches from said item constructed from a fabric at a temperature of approximately 356° F. for approximately 6 minutes;
dispersing ozone around said item constructed from a fabric;
vacuuming said item constructed from a fabric with a plurality of vacuums which are nearly touching said item constructed from a fabric and which produce a volumetric air flow of approximately 1280 cubic feet per minute each;
heating for approximately 6 minutes said item constructed from a fabric with infrared heat of approximately 265° F. located approximately 18 inches away from said item constructed from a fabric for approximately 6 minutes;
vacuuming said item constructed from a fabric with a plurality of vacuums which are nearly touching said item constructed from a fabric and which produce a volumetric air flow of approximately 1280 cubic feet per minute each.

37. The method for sanitizing and cleaning items constructed from a fabric as defined in claim 36 wherein said item constructed from a fabric is a pillow.

38. The method for sanitizing and cleaning items constructed from a fabric as defined in claim 36 wherein said item constructed from a fabric is a seat cushion.

39. The method for sanitizing and cleaning items constructed from a fabric as defined in claim 36 wherein said item constructed from a fabric is a blanket.

40. The method for sanitizing and cleaning items constructed from a fabric as defined in claim 36 wherein said item constructed from a fabric is a pet bed.

41. The method for sanitizing and cleaning items constructed from a fabric as defined in claim 36 wherein said item constructed from a fabric is a rug.

42. The method for sanitizing and cleaning items constructed from a fabric as defined in claim 36 wherein said item constructed from a fabric is an article of clothing.

43. The method for sanitizing and cleaning items constructed from a cloth fabric as defined in claim 36 wherein said item constructed from a cloth fabric is an article of luggage.
